(19) United States
(12) Patent Application Publication
(10) Pub. No.: US 2009/0169501 A1
(43) Pub. Date: Jul. 2, 2009

(54) MASCARA CONTAINING A SUGAR SILICONE SURFACTANT

(75) Inventors: Sue Feng, Edison, NJ (US); Lilavati Patel, Edison, NJ (US); Christi Kagiyama, Tokyo (JP)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL S.A, Paris (FR)

(21) Appl. No.: 12/343,691

(22) Filed: Dec. 24, 2008

Related U.S. Application Data
(60) Provisional application No. 61/017,368, filed on Dec. 28, 2007.

(51) Int. Cl. A61K 8/60; A61Q 1/10

(52) U.S. Cl. 424/70.7

(57) ABSTRACT

The invention relates to a mascara composition comprising at least one sugar silicone surfactant, as well as to methods of making and using such mascaras.

This application claims priority to U.S. provisional application 61/017,368 filed Dec. 28, 2007, the entirety of which is hereby incorporated by reference.

### FIELD OF THE INVENTION

The present invention generally relates to mascaras comprising at least one sugar silicone surfactant. Such compositions, when applied to eyelashes, allow for smooth application and increased comfort on the lashes. Such compositions can also possess improved properties and characteristics such as, for example, improved lift and curl properties.

### DISCUSSION OF THE BACKGROUND

Many mascaras have been developed for improved wear, transfer-resistance, and length/volume properties. Such properties are typically accomplished by the use of ingredients that form a film after application. Such compositions generally contain volatile solvents, which evaporate on contact with the eyelashes, leaving behind a layer comprising waxes and/or resins, pigments, fillers, and actives. However, generally speaking, mascaras can have stability and other problems, causing clumping of the product which leads to uneven application, uncomfortable or unpleasant application, and uncomfortable or unpleasant feel after application. Thus, there remains a need for improved mascaras which possess improved application and feel properties, particularly those which have other improved cosmetic properties as well.

Accordingly, one aspect of the present invention is a mascara having improved application and feel properties.

### SUMMARY OF THE INVENTION

The present invention relates to mascaras comprising at least one sugar silicone surfactant.

The present invention relates to mascaras comprising at least one sugar silicone surfactant, at least one alkoxylated surfactant having at least 8 carbon atoms and at least two alkoxylation units, and at least one alcohol.

The present invention further relates to methods of making-up eyelashes comprising applying an eyelash making-up effective amount of a composition of the present invention to eyelashes in need of such making-up.

The present invention also relates to methods of treating or caring for eyelashes by applying compositions of the present invention to the eyelashes in an amount sufficient to treat and/or care for the eyelashes.

The present invention further relates to methods of enhancing the appearance of eyelashes by applying compositions of the present invention to the eyelashes in an amount sufficient to enhance the appearance of the eyelashes.

The present invention also relates to methods of making a composition comprising combining an emulsion comprising at least one sugar silicone surfactant, at least one alkoxylated surfactant having at least 8 carbon atoms and at least two alkoxylation units, and at least one alcohol, with at least one other ingredient to form a mascara.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

“Waterproof” as used herein refers to the ability to repel water and permanence with respect to water. Waterproof properties may be evaluated by any method known in the art for evaluating such properties. For example, a mascara composition may be applied to false eyelashes, which may then be placed in water for a certain amount of time, such as, for example, 20 minutes. Upon expiration of the pre-ascertained amount of time, the false eyelashes may be removed from the water and passed over a material, such as, for example, a sheet of paper. The extent of residue left on the material may then be evaluated and compared with other compositions, such as, for example, commercially available compositions. Similarly, for example, a composition may be applied to skin, and the skin may be submerged in water for a certain amount of time. The amount of composition remaining on the skin after the pre-ascertained amount of time may then be evaluated and compared. For example, a composition may be waterproof if a majority of the product is left on the wearer, e.g., eyelashes.

“Tackiness” as used herein refers to the adhesion between two substances. For example, the more tackiness there is between two substances, the more adhesion there is between the substances. To quantify “tackiness,” it is useful to determine the “work of adhesion” as defined by IUPAC associated with the two substances. Generally speaking, the work of adhesion measures the amount of work necessary to separate two substances. Thus, the greater the work of adhesion associated with two substances, the greater the adhesion there is between the substances, meaning the greater the tackiness is between the two substances.

Work of adhesion and, thus, tackiness, can be quantified using acceptable techniques and methods generally used to measure adhesion, and is typically reported in units of force time (for example, gram seconds (“g s”)). For example, the TA-XT2 from Stable Micro Systems, Ltd. can be used to determine adhesion following the procedures set forth in the TA-XT2 Application Study (ref: MATI/PO.25), revised January 2000, the entire contents of which are hereby incorporated by reference. According to this method, desirable values for work of adhesion for substantially non-tacky substances include less than about 0.5 g s, less than about 0.4 g s, less than about 0.3 g s and less than about 0.2 g s. As known in the art, other similar methods can be used on other similar analytical devices to determine adhesion.

The cosmetic compositions and methods of the present invention can comprise, consist of, or consist essentially of the essential elements and limitations of the invention described herein, as well as any additional or optional ingredients, components, or limitations described herein or any otherwise useful ingredient found in personal care compositions intended for application to keratin materials.

The composition of the present invention may be in any form suitable for use on eyelashes such as, for example, emulsion compositions (for example, water-in-oil emulsion, oil-in-water emulsion, multiple emulsion (W/O/W or O/W/O), nanoemulsions, etc.). Generally speaking, mascaras contain colorants such as pigments. Additionally, the compositions of the present invention can be clear or transparent: that is, they can contain little or no colorants. The compositions of the present invention, particularly those with little or no colorants, can be used as a basecoat and/or topcoat for application beneath and/or onto other products applied to eyelashes.

As defined herein, stability is tested by placing the composition in a controlled environment chamber for 8 weeks at 25° C. In this test, the physical condition of the sample is inspected as it is placed in the chamber. The sample is then inspected again at 24 hours, 3 days, 1 week, 2 weeks, 4 weeks and 8 weeks. At each inspection, the sample is examined for abnormalities in the composition such as phase separation if the composition is in the form of an emulsion. The stability is further tested by repeating the 8-week test at 40° C., 37° C., 45° C., 50° C. and/or under freeze-thaw conditions. A composition is considered to lack stability if in any of these tests an abnormality that impedes functioning of the composition is observed. The skilled artisan will readily recognize an abnormality that impedes functioning of a composition based on the intended application.

Sugar Silicone Surfactant

According to the present invention, compositions comprising at least one sugar silicone surfactant are provided. The sugar silicone surfactant of the present invention has the following formula:

Sach-X-Dₙ-X-Sach

where Sach represents a saccharide moiety containing multiple hydroxyl groups. Suitable saccharide moieties include, but are not limited to, those based on monosaccharides such as, for example, glucose, fructose, galactose, ribose, mannose, sorbose, etc., and those based one oligosaccharides such as, for example, sucrose, lactose, palatinose, raffinose, lactosucrose, glucosylsucrose, galactosyl-sucrose, xylobiose, etc. Preferably, the saccharide moiety is based on a monosaccharide, most preferably glucose;

X represents a linear or branched, saturated or unsaturated, C₁ to C₄₀ hydrocarbon-based group, possibly containing in their chain one or more oxygen, sulphur and/or nitrogen atoms. Preferably, X represents a linear, unsubstituted alkyl group containing at least one N atom, most preferably a linear, unsubstituted alkyl group having 1-6 carbon atoms and at least one N atom;

D represents a silicone based group of the formula R₂SiO, where R₂ represents a linear or branched, saturated or unsaturated, C₁ to C₁₀ hydrocarbon-based group. Preferably, R₂ is an unsubstituted C₁ to C₃ alkyl group (methyl, ethyl, propyl), most preferably a methyl group; and

n represents a number between 1 and 1000, preferably between 100 and 500, more preferably between 250 and 400, and more preferably between 300 and 350, including all ranges and subranges therebetween.

Preferably, such sugar silicone surfactants are prepared by reacting a lactone form of the saccharide with an amino form of the D group, thereby forming an alkyl group X having an N atom between the saccharide moiety and the silicone moiety.

Particularly preferred sugar silicone surfactants include gluconamidoethylaminopropylsilicone, lactobionolactonesiloxane, or a mixture thereof.

Preferably, the sugar silicone surfactant represents from about 0.5% to about 20% of the total weight of the composition, more preferably from about 0.75% to about 15% of the total weight of the composition, and most preferably from about 1% to about 10%, including all ranges and subranges therebetween.

Alkoxylated Surfactant

According to preferred embodiments of the present invention, compositions of the present invention further comprise at least one alkoxylated surfactant having at least 8 carbon atoms and at least two alkoxylation units. Preferably, the alkoxylated surfactant comprises 8 to 30 carbon atoms, more preferably 8 to 22 carbon atoms, and most preferably 10 to 16 carbon atoms. Also preferably, the alkoxylated surfactants comprise 2 to 100 alkoxylation units, more preferably 10 to 50 alkoxylation units, and most preferably 20 to 50 alkoxylation units. Suitable alkoxylated surfactants are well known in the art and comprise fatty potions (for example, cetyl, stearyl, oleyl, synthetic fatty alcohols, etc.) in combination with alkoxylation units (for example, ethoxylation and propoxylation). Specific examples include, but are not limited to, steareth surfactants (for example, steareth-21), ceteth surfactants, oleth surfactants, pareth surfactants and the like. A particularly preferred surfactant is C11-C15 Pareth-40.

Preferably, the alkoxylated surfactant having at least 8 carbon atoms and at least two alkoxylation units represents from about 0.5% to about 20% of the total weight of the composition, more preferably from about 0.75% to about 15% of the total weight of the composition, and most preferably from about 1% to about 10%, including all ranges and subranges therebetween.

Alcohol

According to the present invention, compositions comprising at least one alcohol are provided. The alcohol may be either liner or branched. Preferably, the alcohol is non-volatile, having an elevated boiling point. For example, the alcohol has a boiling point of at least 50° C., 75° C., or 100° C. Also preferably, the alcohol has at least 8 carbon atoms, preferably at least 10 carbon atoms. Suitable examples of acceptable alcohols include butyl octanol, butyl nonanol, butyl decanol, pentyl octanol, pentyl nonanol, pentyl decanol, hexyl octanol, hexyl nonanol, and hexyl decanol.

Preferably, the alcohol(s) represent from about 0.1% to about 30% of the total weight of the composition, more preferably from about 0.5% to about 15% of the total weight of the composition, and most preferably from about 1% to about 10%, including all ranges and subranges therebetween.

According to particularly preferred embodiments of the present invention, the alcohol, the alkoxylated surfactant having at least 8 carbon atoms and at least two alkoxylation units, and the sugar silicone surfactant are combined to form an emulsion. A suitable example of such an emulsion is the product sold by Dow Corning under the designation CE-8810 (gluconamidoethylaminopropylsilicone/C11-C15 Pareth-40/butyloctanol).

Viscosity Increasing Agents/Film Forming Agents The composition may also contain at least one viscosity increasing agent and/or film forming agent. Preferably, the compositions of the present invention include one or more viscosity increasing agents and/or film forming agents. Suitable examples of viscosity increasing agents and/or film forming agents include the following.

Elastomeric compounds such as those sold or made under the names KSG6 from Shin-Etsu, Trefil E-505C or Trefil E-506C from Dow-Corning, Gransil from Grant Industries (SR-CYC, SR DMF10, SR-DC556) or those marketed in the form of preconstituted gels (KSG15, KSG17, KSG16, KSG18, KSG21 from Shin-Etsu, Gransil SR 5CYC gel, Gransil SR DMF 10 gel, Gransil SR DC556 gel, SF 1204 and JK 113 from General Electric or emulsifying elastomers such as those sold under the names of KSG-210, KSG-30, KSG-31, KSG-32, KSG-33, KSG-40, KSG 41, KSG-42, KSG-43 and KSG-44 from Shin-Etsu.

Bimodal film forming agents which form a bimodal interpenetrating network containing multiple functionalities (for example, cationic and anionic functionalities) which is reversibly cross-linked at least partially through the multiple functionalities. Exemplary bimodal film forming agents are disclosed in PCT patent application nos. WO 05/087191 and WO 06/028931, and corresponding U.S. provisional application Nos. 60/551,658, 60/606,985, and 60/627,224, the entire contents of all of which are hereby incorporated by reference in their entirety. Suitable bimodal film forming agents include, but are not limited to, film forming agents having both cationic and anionic functionalities. According to particularly preferred embodiments of the present invention, the bimodal film forming agent comprises at least one acrylic acid-based, (meth)acrylic acid-based, acrylate-based or (meth)acrylate-based monomer having anionic and/or cationic functionalities. Suitable polymers or copolymers include, but are not limited to, polymers comprising polyacrylates such as those identified in the International Cosmetic Ingredient Dictionary and Handbook (9^{th} ed. 2002) such as, for example, polyacrylate-1, polyacrylate-2, polyacrylate-3, polyacrylate-4 . . . polyacrylate-16, polyacrylate-17, polyacrylate-18, polyacrylate-19 . . . , etc. Such (co)polymers, or similar (co)polymers, can be combined individually or with other (co)polymers in such a way to form suitable bimodal film forming agents having both cationic and anionic functionalities. According to particularly preferred embodiments, the bimodal film forming agent is selected from the group consisting of polymers consisting of polyacrylate-21 and acrylates/dimethylaminoethylmethacrylate copolymer (marketed under the name Syntran PC 5100 by Interpolymer), polyacrylate-16 (marketed under the name Syntran PC 5112 by Interpolymer), polyacrylate-18 and polyacrylate-19 (marketed under the name Syntran PC 5107 by Interpolymer), and polyacrylate-18 and polyacrylate-1 g (marketed under the name Syntran PC 5117 by Interpolymer). The bimodal film forming agent containing polyacrylate-21 and acrylates/dimethylaminoethylmethacrylate copolymer (Syntran PC 5100) and polyacrylate-16 (Syntran PC 5112) are particularly preferred.

Liposoluble or dispersible rheological polymers which increase viscosity of a composition and is compatible with fatty materials. Such polymers, however, are not waxes. Suitable examples of acceptable liposoluble polymers include, but are not limited to, polyalkylenes, in particular polybutene, poly(meth)acrylates, alkylcelluloses with a linear or branched, saturated or unsaturated C₁ to C₈ alkyl radical, such as ethylcellulose and propylcellulose, silicone polymers that are compatible with the fatty phase, polyvinylpyrrolidone (PVP) or vinylpyrrolidone (VP) homopolymers or copolymers, copolymers of a C₂ to C₃₀, such as C₃ to C₂₂ alkene, and combinations thereof. As specific examples of VP copolymers which can be used in the invention, mention may be made of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate copolymer.

Polar modified waxes which are made using metallocene catalysis which have been modified to include polar groups or units. Suitable polar modified waxes include those disclosed in U.S. patent application publication no. 20070031361, the entire contents of which is hereby incorporated by reference. Preferably, the polar modified wax is based upon a homopolymer and/or copolymer wax of ethylene and/or propylene monomers having a weight-average molecular weight Mw of less than or equal to 25 000 g/mol, preferably of 1000 to 22 000 g/mol and particularly preferably of 4000 to 20,000 g/mol, a number-average molecular weight Mn of less than or equal to 15 000 g/mol, preferably of 500 to 12 000 g/mol and particularly preferably of 1000 to 5000 g/mol, a molar mass distribution Mw/Mn in the range from 1.5 to 10, preferably from 1.5 to 5, particularly preferably from 1.5 to 3 and especially preferably from 2 to 2.5, which have been obtained by metallocene catalysis. In the case of a copolymer wax, it is preferable to have, based on the total weight of the copolymer wax, 0.1 to 30.0% by weight of structural units originating from the one monomer and 70.0 to 99.9% by weight of structural units originating from the other monomer. Such homopolymer and copolymer waxes can be made, for example, by the process described in EP 571 882, the entire contents of which is hereby incorporated by reference, using the metallocene catalysts specified therein. Suitable preparation processes include, for example, suspension polymerization, solution polymerization and gas-phase polymerization of olefins in the presence of metallocene catalysts, with polymerization in the monomers also being possible.

Polar modified waxes can be produced in a known manner from the homopolymers and copolymers described above by oxidation with oxygen-containing gases, for example air, or by graft reaction with polar monomers, for example maleic acid or acrylic acid or derivatives of these acids. The polar modification of metallocene polyolefin waxes by oxidation with air is described, for example, in EP 0 890 583 A1, and the modification by grafting is described, for example, in U.S. Pat. No. 5,998,547, the entire contents of both of which are hereby incorporated by reference in their entirety.

In accordance with the present invention, particularly preferred polar modified waxes are hydrophilically modified waxes (that is, waxes which have been modified to provide the waxes with hydrophilic properties). Suitable examples include, but are not limited to, homopolymers and copolymers of hydrophobic groups such as C2-C30 groups, for example ethylene, propylene, C18-C30, etc. which have been modified with hydrophilic units such as, for example, maleic anhydride, acrylate, methacrylate, polyvinylpyrrolidone (PVP), etc. Particularly preferred hydrophilically modified waxes are ethylene and propylene homopolymers and copolymers which have been modified with acrylate and/or maleic anhydride units.

In accordance with the present invention, particularly preferred polar modified waxes also include hydrophobically modified waxes (that is, waxes which have been modified to provide the waxes with hydrophobic properties). Suitable examples include, but are not limited to, hyrdrophilic homopolymers and copolymers of maleic anhydride, acrylate, methacrylate, polyvinylpyrrolidone (PVP), etc. which have been modified with hydrophobic groups such as C2-C30 groups, for example ethylene, propylene, C18-C30, etc.

Particularly preferred polar modified waxes for use in the present invention are polypropylene-maleic anhydride modified waxes (“PPMA”) commercially available from Clariant under the trade name LICOCARE, and C26-C28 alpha olefin maleic acid anhydride copolymer modified waxes. Specific examples of such waxes include products marketed by Clariant under the LicoCare name having designations such as PP207 LP3349, CM401 LP3345, CA301 LP 3346, and CA302 LP 3347.

The composition may also contain modified clays such as, for example, hectorites modified with an ammonium chloride of a C₁₀ to C₂₂ fatty acid, such as hectorite modified with distearyidimethylammonium chloride, also known as quatermium-18 bentonite, such as the products sold or made under the names Bentone 34 by the company Rheox, Claytone XL, Claytone 34 and Claytone 40 sold or made by the company Southern Clay, the modified clays known under the name quaternium-18 benzalkonium bentonites and sold or made under the names Claytone HT, Claytone GR and Claytone PS by the company Southern Clay, the clays modified with stearyidimethylbenzoylammonium chloride, known as steralkonium bentonites, such as the products sold or made under the names Claytone APA and Claytone AF by the company Southern Clay, and Baragel 24 sold or made by the company Rheox.

The composition may also contain silica, such as fumed silica. The fumed silica may have a particle size, which may be nanometric to micrometric, for example ranging from about 5 nm to 200 nm.

The fumed silicas may be obtained by high-temperature hydrolysis of a volatile silicon compound in a hydrogen-oxygen flame, producing a finely divided silica. This process makes it possible to obtain hydrophilic silicas that have a large number of silanol groups at their surface. Such hydrophilic silicas are sold or made, for example, under the names “Aerosil 130®”, “Aerosil 200®”, “Aerosil 255®”, “Aerosil 300®” and “Aerosil 380®” by the company Degussa, and “CAB-O-SIL HS-55®”, “CAB-O-SIL EH-5®”, “CAB-O-SIL LM-130®”, “CAB-O-SIL MS-55®” and “CAB-O-SIL M-5®” by the company Cabot.

It is thus possible to chemically modify the surface of the hydrophilic silica by chemical reaction, producing a reduction in the number of silanol groups. The silanol groups can be replaced, for example, with hydrophobic groups: this then gives a hydrophobic silica. The hydrophobic groups may be (a) trimethylsiloxyl groups, which are obtained in particular by treating fumed silica in the presence of hexamethyldisilazane. Silicas thus treated are known as “silica silylate” according to the CTFA (6th edition, 1995). They are sold or made, for example, under the references “Aerosil R812®” by the company Degussa and “CAB-O-SIL TS-530®” by the company Cabot; (b) dimethylsilyloxyl or polydimethylsiloxane groups, which are obtained in particular by treating fumed silica in the presence of polydimethylsiloxane or dimethyldichlorosilane. Silicas thus treated are known as “silica dimethyl silylate” according to the CTFA (6th edition, 1995). They are sold or made, for example, under the references “Aerosil R972®” and “Aerosil R974®” by the company Degussa, and “CAB-O-SIL TS-610®” and “CAB-O-SIL TS-720®” by the company Cabot; and (c) groups derived from reacting fumed silica with silane alkoxides or siloxanes. These treated silicas are, for example, the products sold or made under the reference “Aerosil R805®” by the company Degussa.

Using hydrophobic silicas, such as fumed silica, makes can help in obtaining a translucent or even transparent composition, in particular in the form of a stick, which does not exude, in the absence of opacifying particles such as waxes, fillers and pigments (including nacres).

The composition may also contain at least one typical wax. For the purposes of the present invention, a typical wax is a lipophilic fatty compound that is solid at room temperature (25° C.) and atmospheric pressure (760 mmHg, i.e. 101 KPa), which undergoes a reversible solid/liquid change of state, having a melting point of greater than 40° C. and further such as greater than 55° C. and which may be up to 200° C. By bringing the wax to its melting point, it is possible to make it miscible with oils and to form a microscopically homogeneous mixture, but on returning the temperature of the mixture to room temperature, recrystallization of the wax in the oils of the mixture is obtained. It is this recrystallization in the mixture which is responsible for the reduction in the gloss of the mixture.

For the purposes of the invention, the waxes are those generally used in cosmetics and dermatology; they are, for example, of natural origin, for instance beeswax, ozokerite, carnauba wax, candelilla wax, ouricury wax, Japan wax, cork fiber wax, sugar cane wax, paraffin wax, lignite wax, microcrystalline waxes, lanolin wax, montan wax, ozokerites and hydrogenated oils such as hydrogenated jojoba oil as well as waxes of synthetic origin, for instance polyethylene waxes derived from the polymerization of ethylene, waxes obtained by Fischer-Tropsch synthesis, fatty acid esters and glycerides that are solid at 40° C., for example, at above 55° C., fatty alcohol waxes such as those sold by Baker Petrolite under the Performacol name (Performacol 350, 425 and 550) including C30-C50 alcohols, silicone waxes such as alkyl- and alkoxy-poly(di)methylsiloxanes and/or poly(di)methyl-siloxane esters that are solid at 40° C., for example, at above 55° C.

According to the invention, the melting point values correspond to the melting peak measured by the “Differential Scanning Calorimetry” method with a temperature rise of 5 or 1° C./min.

The composition may also include a polysilicone-polyamide copolymer such as those disclosed in U.S. patent application publication no. 2004/0170586 (a particularly preferred type of copolymer being a nylon 611/dimethicone copolymer), a tackifier (for example, those sold under the Regalite name), and/or a block copolymer such as an A-B or an A-B-A type copolymer (for example, those sold under the Kraton name).

Other acceptable film forming agents include, but are not limited to, those disclosed in U.S. patent application publication no. 2004/0170586, the entire contents of which is hereby incorporated by reference. Non-limiting representative examples of such other film forming agents include silicone resins such as, for example, MQ resins (for example, trimethylsiloxysilicates), T-propyl silsesquioxanes and MK resins (for example, polymethylsilsesquioxanes), silicone esters such as those disclosed in U.S. Pat. Nos. 6,045,782, 5,334,737, and 4,725,658, the disclosures of which are hereby incorporated by reference, polymers comprising a backbone chosen from vinyl polymers, methacrylic polymers, and acrylic polymers and at least one chain chosen from pendant siloxane groups and pendant fluorochemical groups such as those disclosed in U.S. Pat. Nos. 5,209,924, 4,693,935, 4,981,903, 4,981,902, and 4,972,037, and WO 01/32737, the disclosures of which are hereby incorporated by reference, polymers such as those described in U.S. Pat. No. 5,468,477, the disclosure of which is hereby incorporated by reference (a non-limiting example of such polymers is poly(dimethylsiloxane)-g-poly(isobutyl methacrylate), which is commercially available from 3M Company under the tradename VS 70 IBM).

According to preferred embodiments, the film former and/or viscosity increasing agent, when present, is present in the composition in an amount ranging from 0.1% to 50% by weight relative to the total weight of the composition. Preferably, the film former/viscosity increasing agent is present in an amount ranging from 0.5% to 40% by weight relative to the total weight of the composition, and more preferably from 5% to 30%, including all ranges and subranges therebetween. One of ordinary skill in the art will recognize that the film former/viscosity increasing agent of the present invention may be commercially available, and may come from suppliers in the form of a dilute solution. The amounts of the film former/viscosity increasing agent disclosed herein therefore reflect the weight percent of active material.

Volatile Oil

According to one embodiment of the present invention, compositions optionally further comprising at least one volatile oil are provided. Preferably, the at least one volatile oil is a silicone volatile oil, a hydrocarbon volatile oil, or a mixture thereof.

According to preferred embodiments, the composition may contain one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |
| PDMS DC 200 (5 cSt) from Dow Corning | 134 | 5 |
| PDMS DC 200 (3St) from Dow Corning | 102 | 3 |

Further, a volatile linear silicone oil may be employed in the compositions of the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to other preferred embodiments, the composition may contain one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, isohexadecane, and for example, the oils sold under the trade names of Isopar or Permethyl, the C₈ to C₁₆ branched esters such as isohexyl or isodecyl neopentanoate and their mixtures. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Isohexadecane | 102 |
| Isodecyl Neopentanoate | 118 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

The volatility of the solvents/oils can be determined using the evaporation speed as set forth in U.S. Pat. No. 6,338,839.

Preferably, the volatile oil(s), when present, represent from about 5% to about 90% of the total weight of the composition, more preferably from about 10% to about 80% of the total weight of the composition, and most preferably from about 20% to about 75%, including all ranges and subranges therebetween.

Notwithstanding the above, a preferred embodiment of the present invention is a composition which is substantially free of volatile oil (that is, less than 3% of volatile oil) or free of volatile oil (that is, less than 1% volatile oil).

Coloring Agents

According to the present invention, the compositions may optionally comprise at least one coloring agent (colorant). Suitable coloring agents include but are not limited to pigments, dyes, such as liposoluble dyes, nacreous pigments, and pearling agents. Typically, when the composition contains colorants, it is a make-up composition such as a mascara composition. Alternatively, when the composition does not contain colorants, it is a clear or transparent composition which can be used as a basecoat (or topcoat) prior to (or after) application of a make-up composition such as a mascara to eyelashes, or it can be used as a hair treatment composition such as, for example, a hair conditioner or mousse. However, it is possible that topcoats, basecoats, hair treatment products and the like could contain colorants, and/or that a mascara or make-up composition could contain little or no colorant.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.1% to 20%, preferably from 0.1% to 15%.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the pigments may be present in the composition in a concentration ranging up to 50% by weight of the total weight of the composition, such as from 0.5% to 40%, and further such as from 2% to 30%. In the case of certain products, the pigments, including nacreous pigments, may, for example, represent up to 50% by weight of the composition.

According to preferred embodiments of the present invention, the compositions of the present invention further comprise water. In this embodiment, water is preferably present in an amount ranging from about 0.6 to about 70%, preferably from about 3.0 to 60%, and more preferably from about 5 to about 50% relative to the total weight of the composition. Preferably, such water-containing cosmetic compositions are mascaras, and are emulsions or dispersions.

According to particularly preferred embodiments, the compositions of the present invention are in the form of an emulsion. Suitable emulsion forms include but are not limited to oil-in-water, water-in-oil, oil-in-water-in-oil, water-in-oil-in-water and nanoemulsions (emulsions whose oil globules are of very fine particle size, that is to say that they have a number-average size of less than about 100 nanometers (nm)). Emulsions contain at least one oil phase and at least one aqueous phase. Typically speaking, emulsions contain surfactants or surfactant-like materials which provide stability to the emulsions and inhibit de-phasing of the emulsions.

Additional Additives

The composition of the invention can also comprise any additive usually used in the field under consideration. For example, dispersants such as poly(12-hydroxystearic acid), antioxidants, essential oils, sunscreens, preserving agents, fragrances, fillers, neutralizing agents, cosmetic and dermatological active agents such as, for example, emollients, moisturizers, vitamins, essential fatty acids, surfactants, pasty compounds and mixtures thereof can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9^{th} ed. 2002).

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable medium and should be able to be applied to the eyelashes of human beings.

Non-limiting examples of such additional components include non-volatile oils such as silicone oils (for example, dimethicone, phenyl trimethicone, trimethyl pentaphenyl trisiloxane, etc) or hydrocarbon oils (for example, esters). In one embodiment of the present invention, the compositions of the present invention are substantially free of silicone oils (i.e., contain less than about 0.5% silicone oils). In another embodiment, the compositions are substantially free of non-silicone oils (i.e., contain less than about 0.5% non-silicone oils).

According to preferred embodiments of the present invention, methods of increasing eyelash volume, strength and/or length comprising applying to eyelashes an eyelash volume-, strength- and/or length-increasing effective amount of a composition comprising at least one sugar silicone surfactant are provided. Preferably, such compositions further include an alkoxylated surfactant and an alcohol. The compositions may be applied to eyelashes as needed, preferably once or twice daily, more preferably once daily and then preferably allowed to dry before subjecting to contact such as with clothing or other objects.

According to preferred embodiments, methods of increasing the volume, strength or length a composition provides to eyelashes after or upon application to the eyelashes comprising combining at least one sugar silicone surfactant and at least one other ingredient prior to application are provided. Preferably, the at least one sugar silicone surfactant is in the form of an emulsion comprising at least one sugar silicone surfactant, at least one alkoxylated surfactant, and at least one alcohol, and the emulsion is combined with at least one other ingredient to form a mascara. Such a combination of the required elements in a single composition at any time prior to application of the composition to the eyelashes results in increasing of the volume, strength and/or length of the eyelashes after application to the eyelashes.

According to yet further embodiments of the present invention, methods of making-up eyelashes comprising applying an eyelash making-up effective amount of a composition comprising at least one sugar silicone surfactant and at least one coloring agent to eyelashes in need of such making-up are provided. Preferably, such compositions further include an alkoxylated surfactant and an alcohol. Also preferably, “making up” the eyelashes includes applying at least one coloring agent to the eyelashes in an amount sufficient to provide color to the eyelashes.

According to preferred embodiments of the present invention, methods of treating or caring for eyelashes by applying compositions of the present invention to the eyelashes in an amount sufficient to treat and/or care for the eyelashes are provided.

According to other preferred embodiments, methods of enhancing the appearance of eyelashes by applying compositions of the present invention to the eyelashes in an amount sufficient to enhance the appearance of the eyelashes are provided.

In accordance with the preceding preferred embodiments, the compositions of the present invention are applied topically to eyelashes in an amount sufficient to treat, care for and/or make up the eyelashes, or to enhance the appearance of the eyelashes. The compositions may be applied to eyelashes as needed, preferably once or twice daily, more preferably once daily and then preferably allowed to dry before subjecting to contact such as with clothing or other objects.

According to a preferred embodiment of the present invention, compositions having improved cosmetic properties such as, for example, improved feel upon application, and improved feel after application are provided. The improved properties may also be chosen from improved flexibility, wearability, drying time or retention as well as reduced tackiness or migration over time

According to yet other embodiments of the present invention, methods of making a composition comprising mixing together at least one emulsion comprising at least one sugar silicone surfactant, at least one alkoxylated surfactant, and at least one alcohol, and at least one other ingredient to form a mascara composition are provided.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### Example 1

### Mascara

| | |
|---|---|
| Beeswax | 4.00% |
| Carnauba Wax | 3.00% |
| Rice Bran Wax | 7.00% |
| Stearyl Alcohol | 2.50% |
| Water | 43.77% |
| Hydroxyethyl Cellulose | 1.00% |
| Methylparaben | 0.13% |
| Disodium EDTA | 0.10% |
| Simethicone | 0.10% |
| Phenoxyethanol | 0.40% |
| Gluconamidoethylaminopropylsilicone/C11-15 | 10.00% |
| Pareth-40/Butyloctanol (DC CE-8810 | |
| Sugar Siloxane Emulsion) (47.5% sugar | |
| surfactant) | |
| PEG/PG-17/18 Dimethicone | 1.00% |
| Acrylates Copolymer | 5.00% |
| Polyacrylate-21 | 10.00% |
| Acrylates/Dimethylaminoethyl Methacrylate | |
| Copolymer (Syntran PC 5100)(17.5% | |
| active agent) | |
| Black 2 | 12.00% |

### Example 2

### Mascara

| INCI Name | |
|---|---|
| Carnauba Wax | 4.00 |
| Candelilla Wax | 8.00 |
| Jojoba Butter | 4.00 |
| Stearyl Alcohol | 2.50 |
| Beeswax | 4.00 |
| Water | 42.55 |
| Hydroxyethyl Cellulose | 1.00 |
| Methylparaben | 0.25 |
| Disodium EDTA | 0.10 |
| Simethicone | 0.10 |
| Black Iron Oxide | 8.00 |
| Kaolin | 2.00 |
| Gluconamidoethylaminopropylsilicone | 11.00 |
| (DC CE-8810 Sugar Siloxane | |
| Emulsion) (47.5% sugar surfactant) | |
| Phenoxyethanol | 0.50 |
| PPG/IDPI/DMPA Copolymer | 8.40 |
| (Avalure) (38% active agent) | |
| Polyacrylate-21 | 3.60 |
| Acrylates/Dimethylaminoethyl | |
| Methacrylate Copolymer (Syntran PC | |
| 5100) (17.5% active agent) | |

### Example 3

The composition of example 2 was placed into packaging for commercial products “Full and Soft” and “Volume Express” and the thickening properties of this composition in both types of packaging was analyzed. The analysis included comparing thickness values before and after application to lashes, comparing thickness values of the composition of example 2 in both types of packaging, and determining percent (%) volume increase in lashes after application of the composition in both types of packaging. For purposes of this experiment, there were six “lashes” containing five fibers each for a total of thirty fibers made from medium brown, European, “virgin,” hair (DeMeo, Lot # 3-24-00).

Although a total of sixteen (16) lash sets were prepared, twelve (12) [six (6) lash sets per mascara] with similar means and standard deviations were used for the study. A t-test was performed in order to determine if there were any differences. between the baseline values of the two (2) groups for randomization purposes. In addition, a paired “t” test was conducted to compare the changes in the breadth measurements between the corresponding pre-treated and post-treated mascara (Pre-A vs. Post-A) values for each group.

The differences between baseline and post-treated fibers (Post-treatment fibers-Baseline fibers) were calculated for the two (2) groups. A t-test was used to compare the differences between the mascaras.

The increase in volume was calculated using the following: [(r2/rl)²−1]×100=percent (%) increase in volume.

The statistical significance level was set at p−Value≦0.05.

Comparing Thickness Values Before and after Application to Lashes

For randomization purposes, there were no differences found between lash breadth measurements for the two (2) groups at baseline.

Lashes treated with each of the two (2) mascaras were found to be statistically significantly greater than the untreated (baseline) breadth measurements. That is, lashes treated with the composition of example 2 using both Full and Soft packaging (pre-treated thickness of 0.097±0.016; post treated thickness 0.176±0.034) and Volume Express packaging (pre-treated thickness of 0.102±0.021; post treated thickness 0.168±0.034) were statistically significantly thicker than baseline.

Comparing Thickness Values of the Composition of Example 2 in Both Types of Packaging

When comparing the breadth differences between the two (2) groups, there were no statistically significant differences between them.

Determining Percent (%) Volume Increase in Lashes after Application of the Composition in Both Types of Packaging

The greatest increase in volume (245%) was obtained after lashes were treated with the composition of example 2 using Full and Soft packaging. Treating lashes with the composition of example 2 using Volume Express packaging resulted in an increase in volume of 191%.

### Example 4

The composition of example 2 was again placed into packaging for commercial products “Full and Soft” and “Volum' Express.” This time the compositions were compared to the commercial Full and Soft and Volum' Express products, each in its own packaging.

Twelve sensory panelists trained in the application and evaluation of mascara products participated in the study over a four day period. The expert panel is trained to detect small differences not always perceivable by consumers.

Two samples were evaluated each day using a sequential monadic test design. The samples were presented to panelists in an order randomized within each replication block and across panelists. The mascara was applied to the eyelashes using the following protocol.

1. Panelists used a standard soap to wash their face before product application and allowed their skin to equilibrate for 10 minutes.

2. Panelists pump the mascara brush in a scooping manner three times and remove the wand from the package. Tip is wiped off to remove excess mascara.

3. Panelists examine the brush prior to application.

4. The panelists hold the mascara wand with their dominant hand. The wand is held so that the tip of the brush points towards the panelist's nose. The panelist applies 10 strokes of mascara to the lashes. The panelists apply the mascara from inner to outer eye, always from root to tip, rotating the brush after 5 strokes.

5. Step 4 is repeated twice for a total of 30 strokes, re-dipping the brush (one dip) into the mascara packaging to pick up more product each time the step is repeated. The mascara is then evaluated for application, kinesthetic, visual and tactile attributes.

6. The panelists repeat steps 3-5 for a different mascara product on the opposite eye.

Samples were rated using a 15-point universal intensity scale (0=none/15=a lot), using a standard ballot for mascara. The mascaras were evaluated during application to the eye lashes, and immediately after application. After initially rated, the panelists input data into a data entry system. The evaluations were performed in duplicate.

Results

Overall, there were significant differences between the four mascara samples. At application, there were significantly more globs and less evenness of product on the brushes of Full n' Soft and Volum' Express commercial products than there were on the brushes of the composition of example 2 in both types of packaging.

Full n' Soft and Volum' Express commercial products built significantly quicker on the lashes than both samples containing the composition of example 2.

The composition of example 2 in the Volum' Express package had significantly more tugging/drag on lashes during application than the Full n' Soft commercial product.

The Full n' Soft and Volum' Express commercial products had significantly more transfer than both samples containing the composition of example 2.

Visually, Full n' Soft and Volum' Express commercial products provided significantly more length, thickness and curl to the lashes than both Creamy Mascara samples. Full n' Soft and Volum' Express commercial products had significantly more spiking than both samples containing the composition of example 2.

The composition of example 2 in the Full n' Soft package also had significantly more separation than both Full n' Soft and Volum' Express commercial products.

The composition of example 2 in the Full n' Soft package provided a significantly more fanned out look than all other samples. The composition of example 2 in the Volum' Express package had significantly more balls on lashes than Full n' Soft and Volume Express commercial products.

The Full n' Soft and Volum' Express commercial products looked significantly more intense than both Creamy Mascara samples.

What is claimed is:

1. A mascara comprising at least one sugar silicone surfactant.

2. The mascara of claim 1, wherein the sugar silicone surfactant has the following formula
Sach-X-Dₙ-X-Sach
where Sach represents a saccharide moiety containing multiple hydroxyl groups selected from the group consisting of glucose, fructose, galactose, ribose, mannose, sorbose, sucrose, lactose, palatinose, raffinose, lactosucrose, glucosylsucrose, galactosyl-sucrose, xylobiose, and mixtures thereof;
X represents a linear or branched, saturated or unsaturated, C₁ to C₄₀ hydrocarbon-based group, possibly containing in their chain one or more oxygen, sulphur and/or nitrogen atoms;
D represents a silicone based group of the formula R₂SiO, where R₂ represents a linear or branched, saturated or unsaturated, C₁ to C₁₀ hydrocarbon-based group; and
n represents a number between 100 and 500.

3. The mascara of claim 2, wherein Sach represents a glucose moiety.

4. The mascara of claim 3, wherein the sugar silicone surfactant is gluconamidoethylaminopropylsilicone.

5. The mascara of claim 1, further comprising an alkoxylated surfactant and an alcohol.

6. The mascara of claim 5, wherein the alkoxylated surfactant comprises 8 to 30 carbon atoms and 2 to 100 alkoxylation units.

7. The mascara of claim 6, wherein the alcohol has a boiling point greater that 75° C.

8. The mascara of claim 1, wherein the mascara is in the form of an emulsion.

9. The mascara of claim 1, further comprising at least one coloring agent.

10. The mascara of claim 4, further comprising an alkoxylated surfactant and an alcohol.

11. The mascara of claim 10, wherein the alkoxylated surfactant comprises 8 to 30 carbon atoms and 2 to 100 alkoxylation units.

12. The mascara of claim 11, wherein the alcohol has a boiling point greater that 75° C.

13. The mascara of claim 10, wherein the mascara is in the form of an emulsion.

14. The mascara of claim 10, further comprising at least one coloring agent.

15. A method of making up eyelashes comprising applying the composition of claim 1 to the eyelashes.

16. A method of making up eyelashes comprising applying the composition of claim 4 to the eyelashes.

17. A method of making up eyelashes comprising applying the composition of claim 10 to the eyelashes.

18. A method of making up eyelashes comprising applying the composition of claim 11 to the eyelashes.

19. A method of making up eyelashes comprising applying the composition of claim 12 to the eyelashes.

20. A method of making a mascara comprising combining an emulsion comprising at least one sugar silicone surfactant, at least one alcohol and at least one alkoxyalted surfactant with at least one ingredient to form a mascara.
